(19) United States
(12) Patent Application Publication
(10) Pub. No.: US 2026/0020849 A1
(43) Pub. Date: Jan. 22, 2026

(54) SUTURE LOCKING SYSTEMS AND METHODS

(71) Applicant: ARTHREX, INC., Naples, FL (US)

(72) Inventors: Andrew Christian Petry, Naples, FL (US); Allen Holowecky, Naples, FL (US); Thay Q. Lee, Irvine, CA (US); Steven S. Shin, Los Angeles, CA (US)

(21) Appl. No.: 19/272,250

(22) Filed: Jul. 17, 2025

Related U.S. Application Data
(60) Provisional application No. 63/673,888, filed on Jul. 22, 2024.

(51) Int. Cl. A61B 17/04; A61B 17/00; A61B 17/58

(52) U.S. Cl. CPC A61B 17/0401; A61B 17/58; A61B 2017/0088; A61B 2017/00933; A61B 2017/0404; A61B 2017/0414; A61B 2017/044; A61B 2017/0448; A61B 2017/0462

(57) ABSTRACT

Systems and methods are provided for performing tensionable knotless surgical procedures. Suture locking systems that provide one or more one-way locking mechanisms may be utilized for tensioning and locking one or more strands of suture during the surgical procedures. The one-way locking mechanisms may be established by one or more locking barbs or directional fibers provided within the suture locking systems.

### CROSS-REFERENCE TO RELATED APPLICATIONS

This application claims the benefit of U.S. Provisional Application No. 63/673,888, which was filed on Jul. 22, 2024 and is incorporated herein by reference in its entirety.

### BACKGROUND

This disclosure relates to the field of surgery, and more particularly to various suture locking systems that are capable of locking suture when performing a variety of tensionable knotless surgical repairs.

Repetitive trauma to a joint, such as a knee, ankle, hip, elbow, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless surgical procedures, such as tissue repairs, for example. A one-way locking mechanism may be utilized when performing the tensionable knotless surgical procedures for tensioning and locking one or more strands of suture.

An exemplary suture locking system may include, inter alia, a first bone tunnel cap, a second bone tunnel cap configured to engage the first bone tunnel cap, a one-way locking mechanism provided within a cannulation of the first bone tunnel cap, the second bone tunnel cap, or both, and a suture received through the first bone tunnel cap and the second bone tunnel cap. The one-way locking mechanism is configured to allow the suture to be tensioned in a first direction while preventing the suture from moving in a second direction.

Another exemplary suture locking system may include, inter alia, a locking sleeve or a locking screw having an inner diameter wall and an outer diameter wall, a bone engagement feature provided on the outer diameter wall, a plurality of locking barbs protruding inwardly from the inner diameter wall and arranged to establish a one-way locking mechanism, and a suture received through the locking sleeve or the locking screw. The one-way locking mechanism is configured to allow the suture to be tensioned in a first direction while preventing the suture from moving in a second direction.

Yet another exemplary suture locking system may include, inter alia, a suture sheath, a plurality of directional fibers arranged to establish a one-way locking mechanism within a cannulation of the suture sheath, and a suture received through the cannulation.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary suture locking system.

FIG. 2 is a top view of a bone tunnel cap of the suture locking system of FIG. 1.

FIG. 3 is a cross-sectional view through section 3-3 of FIG. 1.

FIG. 4 illustrates a plurality of rows of locking barbs of the bone tunnel cap of FIGS. 2-3.

FIG. 5 illustrates an exemplary suture of the suture locking system of FIG. 1.

FIG. 6 schematically illustrates a surgical method for performing a tissue repair.

FIGS. 7 and 8 illustrate another exemplary suture locking system.

FIGS. 9 and 10 illustrate another exemplary suture locking system.

FIGS. 11 and 12 illustrate yet another exemplary suture locking system.

### DETAILED DESCRIPTION

This disclosure relates to systems and methods for performing tensionable knotless surgical procedures. A suture locking system may include one or more one-way locking mechanisms for tensioning and locking one or more strands of suture during a surgical procedure. These and other features of this disclosure are described in further detail below.

An exemplary suture locking system may include, inter alia, a first bone tunnel cap, a second bone tunnel cap configured to engage the first bone tunnel cap, a one-way locking mechanism provided within a cannulation of the first bone tunnel cap, the second bone tunnel cap, or both, and a suture received through the first bone tunnel cap and the second bone tunnel cap. The one-way locking mechanism is configured to allow the suture to be tensioned in a first direction while preventing the suture from moving in a second direction.

In any further embodiment, the one-way locking mechanism is established by a plurality of locking barbs provided within a cannulation of at least one of the first bone tunnel cap or the second bone tunnel cap.

In any further embodiment, the first bone tunnel cap and the second bone tunnel cap each include a cap portion and a tube portion that protrudes from the cap portion.

In any further embodiment, the tube portion of the first bone tunnel cap or the second bone tunnel cap is accommodated within the tube portion of the other of the first bone tunnel cap or the second bone tunnel cap.

In any further embodiment, the one-way locking mechanism includes a first one-way locking mechanism provided within the first bone tunnel cap and a second one-way locking mechanism provided within the second bone tunnel cap.

In any further embodiment, a hydroxyapatite coating is applied on select portions of the first bone tunnel cap and the second bone tunnel cap.

Another exemplary suture locking system may include, inter alia, a locking sleeve or a locking screw having an inner diameter wall and an outer diameter wall, a bone engagement feature provided on the outer diameter wall, a plurality of locking barbs protruding inwardly from the inner diameter wall and arranged to establish a one-way locking mechanism, and a suture received through the locking sleeve or the locking screw. The one-way locking mechanism is configured to allow the suture to be tensioned in a first direction while preventing the suture from moving in a second direction.

In any further embodiment, the bone engagement feature includes a thread.

In any further embodiment, the suture is connected to a button.

In any further embodiment, the suture includes an eyelet.

In any further embodiment, the suture includes a suture loop.

In any further embodiment, a hydroxyapatite coating is applied on select portions of the outer diameter wall of the locking sleeve or the locking screw.

In any further embodiment, the plurality of locking barbs extend into a cannulation of the locking sleeve or the locking screw.

In any further embodiment, the inner diameter wall circumscribes the cannulation.

Yet another exemplary suture locking system may include, inter alia, a suture sheath, a plurality of directional fibers arranged to establish a one-way locking mechanism within a cannulation of the suture sheath, and a suture received through the cannulation.

In any further embodiment, the one-way locking mechanism allows the suture to be tensioned in a first direction and prevents the suture from moving in a second direction.

In any further embodiment, the plurality of directional fibers are configured to flex relative to the suture sheath.

In any further embodiment, the suture sheath includes an inner diameter wall that circumscribes the cannulation.

In any further embodiment, the plurality of directional fibers protrude inwardly from the inner diameter wall.

In any further embodiment, the suture sheath includes a first stiffness, and the plurality of directional fibers include a second stiffness that is less than the first stiffness.

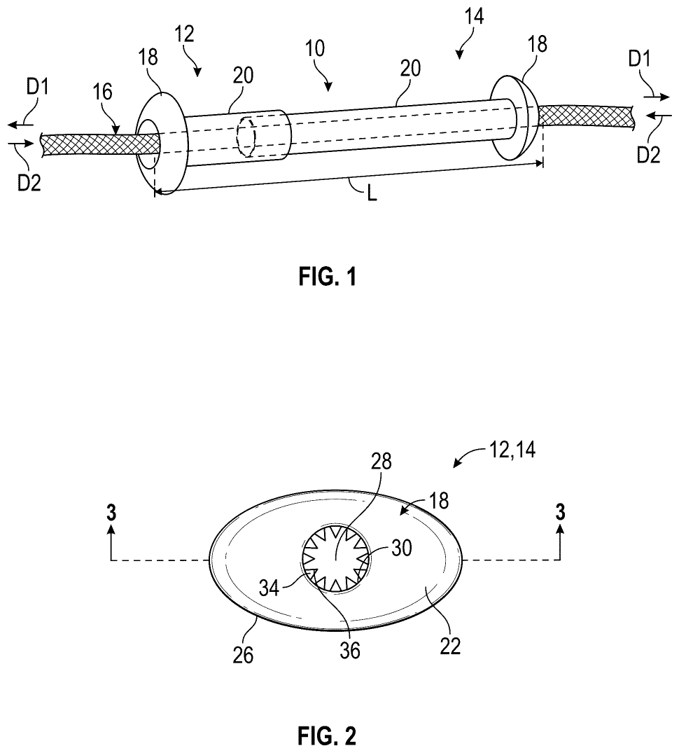

FIGS. 1-4 illustrate various aspects of an exemplary suture locking system 10 that can be used when performing various tensionable knotless surgical procedures. For example, the suture locking system 10 could be utilized during surgical methods for performing tissue repairs. The suture locking system 10 could be used in conjunction with a variety of orthopedic surgical repairs, including but not limited to cerclage procedures, AC joint repairs, syndesmosis repairs, and fracture repairs, among many others.

The suture locking system 10 may include a first bone tunnel cap 12, a second bone tunnel cap 14, and at least one suture 16. The first bone tunnel cap 12 and the second bone tunnel cap 14 may each include a cap portion 18 and a tube portion 20 that protrudes from the cap portion 18. The cap portion 18 and the tube portion 20 may be formed together to establish a unitary structure of each of the first bone tunnel cap 12 and the second bone tunnel cap 14. In an embodiment, the first bone tunnel cap 12 and the second bone tunnel cap 14 are made of a polymer, such as polyetheretherketone (PEEK), for example, or a metallic material, such as a titanium alloy, for example. However, other materials could be utilized within the scope of this disclosure.

The tube portions 20 of the first bone tunnel cap 12 and the second bone tunnel cap 14 may be configured to engage and move relative to one another to adjust a total length L of the suture locking system 10. In an embodiment, the first bone tunnel cap 12 and the second bone tunnel cap 14 may threadably engage one another. For example, the tube portion 20 of one of the first bone tunnel cap 12 or the second bone tunnel cap 14 may include a male thread of a threaded connection between the first and second bone tunnel caps 12, 14, and the tube portion 20 of the other of the first bone tunnel cap 12 or the second bone tunnel cap 14 may include a female thread of the threaded connection for movably connecting the first and second bone tunnel caps 12, 14. In another embodiment, the first bone tunnel cap 12 and the second bone tunnel cap 14 may slidably engage one another. For example, the tube portion 20 of one of the first bone tunnel cap 12 or the second bone tunnel cap 14 may be slightly undersized relative to the tube portion 20 of the other of the first bone tunnel cap 12 or the second bone tunnel cap 14. In such a configuration, the undersized tube portion 20 may slide within the oversized tube portion 20 to adjust the total length L. However, other configurations are contemplated within the scope of this disclosure.

The cap portion 18 of each of the first bone tunnel cap 12 and the second bone tunnel cap 14 may include a top surface 22, a bottom surface 24, and a side wall 26 that extends between the top surface 22 and the bottom surface 24. The bottom surface 24 may include a slightly concave curvature for conforming to the contour of a bone.

The tube portions 20 may protrude outwardly from the bottom surfaces 24 of the cap portions 18. As further detailed below, the first bone tunnel cap 12 and the second bone tunnel cap 14 may each be positioned relative to a bone/bone segment such that the tube portion 20 is accommodated within a socket or tunnel formed in the bone/bone segment, and the cap portion 18 is seated relative to an outer cortex of the bone/bone segment. In the seated position, the top surface 22 of the cap portion 18 faces away from the bone, and the bottom surface 24 faces toward the bone.

A cannulation 28 may extend through the cap portion 18 and the tube portion 20 of each of the first bone tunnel cap 12 and the second bone tunnel cap 14. The cannulations 28 may each establish internal passageways for accommodating one or more strands of the suture 16. When the first bone tunnel cap 12 and the second bone tunnel cap 14 are connected, the tube portion 20 of the second bone tunnel cap 14 is received within the cannulation 28 of the first bone tunnel cap 12 (or vice versa).

Each cannulation 28 may be circumscribed by an interior wall 30 of the first bone tunnel cap 12 or the second bone tunnel cap 14. The interior wall 30 may be established by both the cap portion 18 and the tube portion 20 of the first bone tunnel cap 12 or the second bone tunnel cap 14. An outer diameter wall 32 of each tube portion 20 may provide a smooth surface.

A plurality of locking barbs 34 may protrude inwardly from the interior walls 30. The locking barbs 34 may therefore occupy at least a portion of the open space of the cannulations 28. In an embodiment, the locking barbs 34 are integrally formed (e.g., molded) features of the body of the first bone tunnel cap 12 or second bone tunnel cap 14. The locking barbs 34 may be provided along an entire length of the cannulations 28 or at only select portions thereof. The locking barbs 34 may be either rigid or flexible structures.

The locking barbs 34 may be arranged in multiple rows along the length of the cannulations 28. For example, the locking barbs 34 may be arranged in a least a first row R1 and a second row R2 (see FIG. 4). In an embodiment, the locking barbs 34 of the second row R2 are staggered relative to the locking barbs 34 of the first row R1 (see FIG. 4).

Each locking barb 34 may include a sharp or pointed tip 36, and, in an exemplary implementation, each locking barb 34 may be angled in a direction toward the top surface 22 of the cap portion 18 of each of the first bone tunnel cap 12 and the second bone tunnel cap 14. The locking barbs 34 may therefore establish a one-way locking mechanism that permits one or more sutures 16 to pass through the cannulations 28 in a first direction D1 while preventing the suture(s) 16 from being tensioned or otherwise moved in a second direction D2. The staggered relationship of the rows of locking barbs 34 can maximize engagement with the suture 16 once passed through the cannulations 28.

Although each of the first bone tunnel cap 12 and the second bone tunnel cap 14 may incorporate locking barbs 34 for providing two one-way locking mechanisms within the suture locking system 10, other implementations are contemplated within the scope of this disclosure. For example, in other implementations, only the first bone tunnel cap 12 or only the second bone tunnel cap 14 include the locking barbs 34 for providing a single one-way locking mechanism within the suture locking system 10.

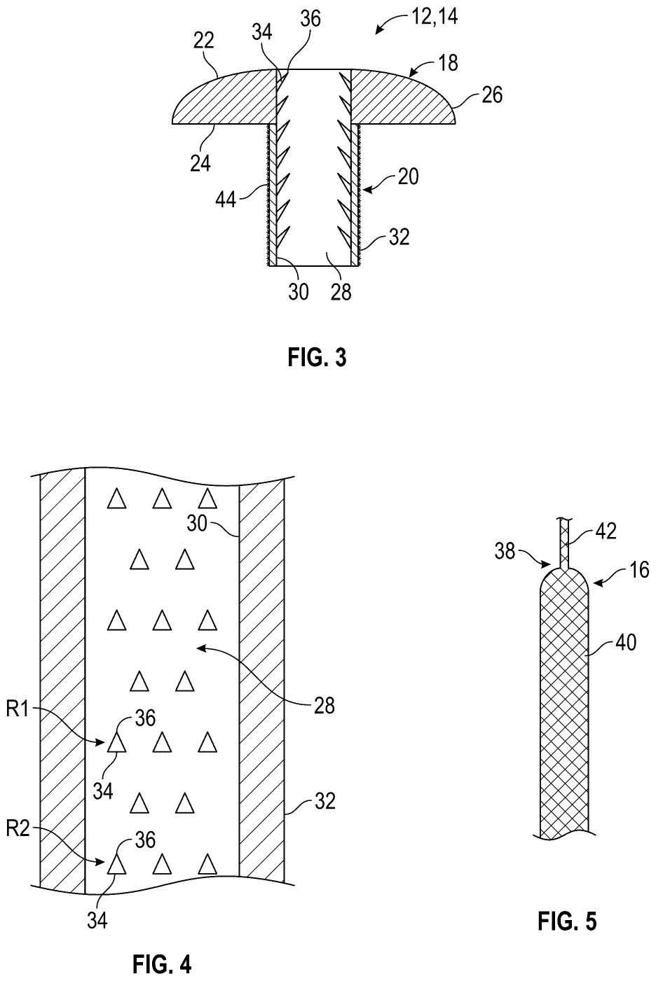

A coating 44 (see FIG. 3) may optionally be applied at select portions of the first bone tunnel cap 12 and the second bone tunnel cap 14. For example, the coating 44 may be applied to the outer diameter wall 32 of each of the first bone tunnel cap 12 and the second bone tunnel cap 14. The coating 44 may be configured to facilitate bony in-growth and thus osteointegration of the suture locking system 10 post implantation. In an embodiment, the coating 44 is a hydroxyapatite coating. However, other types of coatings could also be utilized within the scope of this disclosure.

The suture 16 may be FiberWire®, FiberTape®, or any other suitable suture product. FiberWire® and FiberTape® are suture products marketed and sold by Arthrex, Inc. However, other suture products could be utilized for the suture 16 within the scope of this disclosure. The size and type of suture utilized in conjunction with the suture locking system 10 are not intended to limit this disclosure.

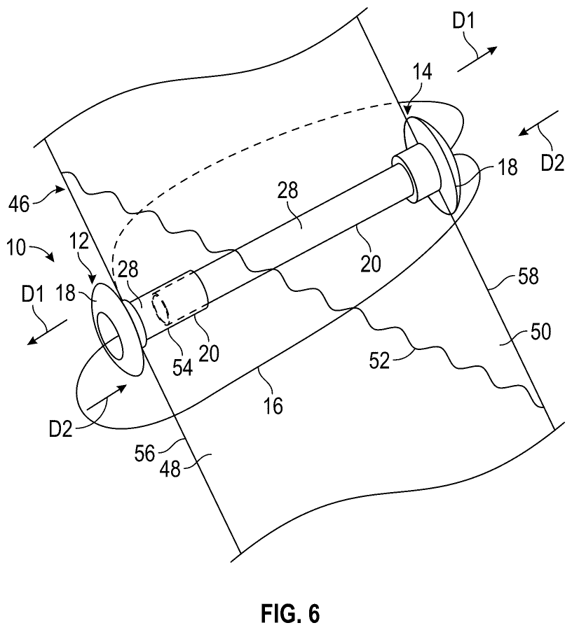

The suture 16 may include a varying thickness. For example, the suture 16 may include one or more tapered regions 38 where the suture 16 transitions between a thickened section 40 and a thinned section 42 (see FIG. 5). The thinned sections 42 can facilitate passing the suture 16 through the cannulations 28, such as via a suitable suture loader that can be provided as part of the suture locking system 10, and the thickened sections 40 can provide greater surface area for the locking barbs 34 to engage in order to sufficiently lock the suture 16 after tensioning the suture 16 relative to the first bone tunnel cap 12 and/or the second bone tunnel cap 14.

In an embodiment, the thickened sections 40 of the suture 16 are about twice as thick as the thinned sections 42. However, other ratios (e.g., 1.5:1, 3:1, etc.) between the relative thicknesses (e.g., outer diameters) of the thickened sections 40 and the thinned sections 42 are contemplated within the scope of this disclosure.

The suture locking system 10 described above and shown in FIGS. 1-5 may be utilized to tension and knotlessly fixate the one or more sutures 16 as part of various surgical methods. FIG. 6 schematically illustrates one such surgical method for repairing a fractured bone 46 as part of a cerclage technique. As schematically illustrated, the fractured bone 46 includes a first bone segment 48 and a second bone segment 50 that are split/separated from one another at a fracture line 52.

After preparing a bone tunnel 54 through the fractured bone 46, the first bone tunnel cap 12 may be inserted into the portion of the bone tunnel 54 formed through the first bone segment 48. Once inserted, the tube portion 20 of the first bone tunnel cap 12 is accommodated within the portion of the bone tunnel 54 formed through the first bone segment 48, and the cap portion 18 of the first bone tunnel cap 12 is seated relative to a cortex 56 of the first bone segment 48. The suture 16 may then be accommodated through the cannulations 28 of both the first bone tunnel cap 12 and the second bone tunnel cap 14.

Next, the second bone tunnel cap 14 may be inserted into the portion of the bone tunnel 54 formed through the second bone segment 50. The tube portion 20 of the second bone tunnel cap 14 may engage the tube portion 20 of the first bone tunnel cap 12 to connect the first and second bone tunnel caps 12, 14 together as the second bone tunnel cap 14 is being inserted into the bone tunnel 54. Once inserted, the tube portion 20 of second first bone tunnel cap 14 is accommodated within the portion of the bone tunnel 54 formed through the second bone segment 50, and the cap portion 18 of the second bone tunnel cap 14 is seated relative to a cortex 58 of the first bone segment 48.

Thereafter, ends of the suture 16 extending from the first bone tunnel cap 12 and the second bone tunnel cap 14 may be wrapped around the fractured bone 46. Each end of the suture 16 may then be tensioned in the first direction D1 to exert a compressive force at each opposing side of the fracture line 52, thereby creating a cerclage for compressing the first and second bone segments 48, 50 together and allowing the fractured bone 46 to properly heal. The locking barbs 34 of each of the first and second bone tunnel caps 12, 14 prevent the suture 16 from moving in the second direction D2 subsequent to the tensioning.

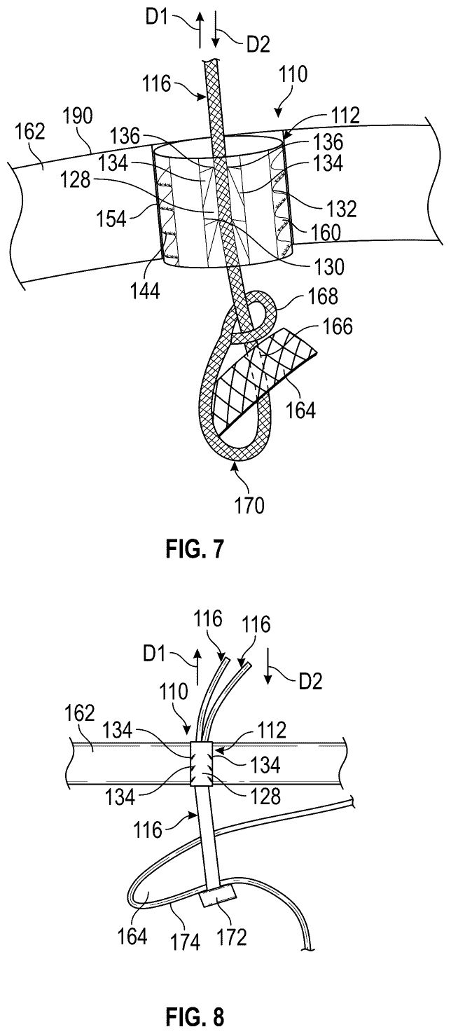

FIG. 7 illustrates another exemplary suture locking system 110 that can be utilized to tension and lock a suture 116 during a surgical procedure. In this embodiment, the suture locking system 110 may be used to secure the suture 116 to a first bone 162 (e.g., a clavicle) and a second bone 164 (e.g., a coracoid), such as part of an acromioclavicular (AC) joint repair, for example.

The suture locking system 110 may include a locking sleeve 112 and one or more strands of the suture 116. In an embodiment, the locking sleeve 112 is made of a polymer, such as PEEK, for example, or a metallic material, such as a titanium alloy, for example. However, other materials could be utilized within the scope of this disclosure.

The locking sleeve 112 may include an inner diameter wall 130 and an outer diameter wall 132. The outer diameter wall 132 may include bone engagement features 160 (e.g., threads, barbs, ledges, etc.) for facilitating fixation to the first bone 162. For example, the locking sleeve 112 may be positioned within a bone tunnel 154 formed in or through the first bone 162 such that the bone engagement features 160 engage a wall of the first bone 162 that circumscribes the bone tunnel 154.

A coating 144 may optionally be applied at select portions of the locking sleeve 112. For example, the coating 144 may be applied to the outer diameter wall 132 and/or the bone engagement feature 160. The coating 144 may be configured to facilitate bony in-growth and thus osteointegration of the suture locking system 110 post implantation. In an embodiment, the coating 144 is a hydroxyapatite coating. However, other types of coatings could also be utilized within the scope of this disclosure.

A cannulation 128 may extend through the locking sleeve 112. The cannulation 128 may establish an internal passageway for accommodating one or more strands of the suture 116. The cannulation 128 may be circumscribed by the inner diameter wall 130. A plurality of locking barbs 134 may protrude inwardly from the inner diameter wall 130. The locking barbs 134 may therefore occupy at least a portion of the open space of the cannulation 128. Each locking barb 134 may include a sharp or pointed tip 136, and, in this implementation, each locking barb 134 may angled in a common direction toward a superior surface 190 of the first bone 162. The locking barbs 134 may therefore establish a one-way locking mechanism that permits the suture 116 to pass through the cannulation 128 in a first direction D1 while preventing the suture 116 from being tensioned or otherwise moved in a second direction D2.

In an embodiment, the suture 116 may be secured to a second bone 164 prior to tensioning and locking the suture 116 in place relative to the first bone 162 via the locking sleeve 112. For example, the suture 116 may be passed through a bone tunnel 166 formed in the second bone 164 and then passed through an eyelet 168 of the suture 116 in order to form a suture loop 170 around the second bone 164. The suture 116 may then be loaded through the cannulation 128 of the locking sleeve 112. The suture 116 may then be tensioned in the direction D1 to allow the pointed tips 136 of the locking barbs 134 to interdigitate with the section of the suture 116 accommodated within the cannulation 128, thereby locking the suture 116 in place and preventing it from sliding back toward the second direction D2. The suture 116 may be passed through the cannulation 128 of the locking sleeve 112 either before or after inserting the locking sleeve 112 into the bone tunnel 154 of the first bone 162. The suture 116 may tensioned until the second bone 164 is located at a desired position relative to the first bone 162.

In another embodiment, shown in FIG. 8, the suture locking system 110 may optionally include a button 172. The button 172 can be utilized in place of the suture loop 170 for achieving fixation of the suture 116 relative to the second bone 164. The suture 116 may be connected to the button 172 prior to passing the button 172 through the bone tunnel 166 and rotating the button 172 until it is seated against an inferior surface 174 of the second bone 164. The suture 116 may subsequently be tensioned and locked in place relative to the first bone 162 via the locking sleeve 112.

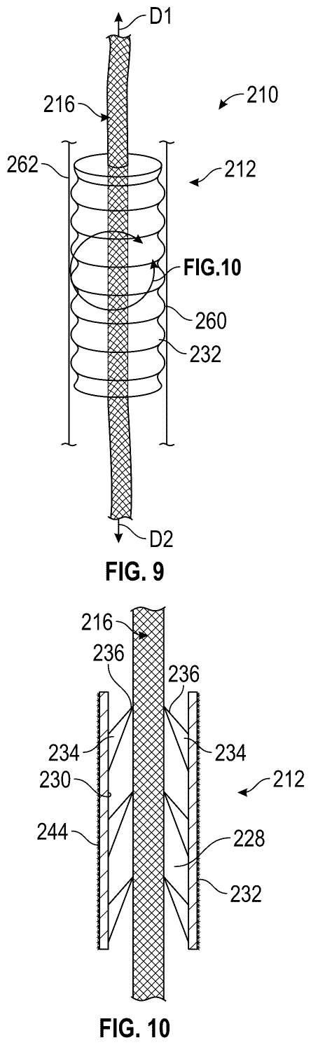

FIGS. 9 and 10 illustrates another exemplary suture locking system 210 that can be utilized to tension and lock a suture 216 during a surgical repair (e.g., soft tissue-to-bone fixation, etc.). The suture locking system 210 may include a locking screw 212 and the suture 216. In an embodiment, the locking screw 212 is made of a polymer, such as PEEK, for example, or a metallic material, such as a titanium alloy, for example. However, other materials could be utilized within the scope of this disclosure.

The locking screw 212 may include an inner diameter wall 230 and an outer diameter wall 232. The outer diameter wall 232 may include an outer thread 260 for inserting the locking screw 212 into an opening formed in a bone 262. A coating 244 may optionally be applied at select portions of the locking screw 212. For example, the coating 244 may be applied to the outer diameter wall 232 and/or the outer thread 260. The coating 244 may be configured to facilitate bony in-growth and thus osteointegration of the suture locking system 210 post implantation. In an embodiment, the coating 244 is a hydroxyapatite coating. However, other types of coatings could also be utilized within the scope of this disclosure.

A cannulation 228 may extend through the locking screw 212. The cannulation 228 may establish an internal passageway for accommodating one or more strands of the suture 216. The cannulation 228 may be circumscribed by the inner diameter wall 230. A plurality of locking barbs 234 (best illustrated in FIG. 10) may protrude inwardly from the inner diameter wall 230. The locking barbs 234 may therefore occupy at least a portion of the open space of the cannulation 228. Each locking barb 234 may include a sharp or pointed tip 236, and, in this implementation, each locking barb 234 may angled in a common direction. The locking barbs 234 may therefore establish a one-way locking mechanism that permits the suture 216 to pass through the cannulation 228 in a first direction D1 while preventing the suture 216 from being tensioned or otherwise moved in a second direction D2 subsequent to tensioning in the first direction D1.

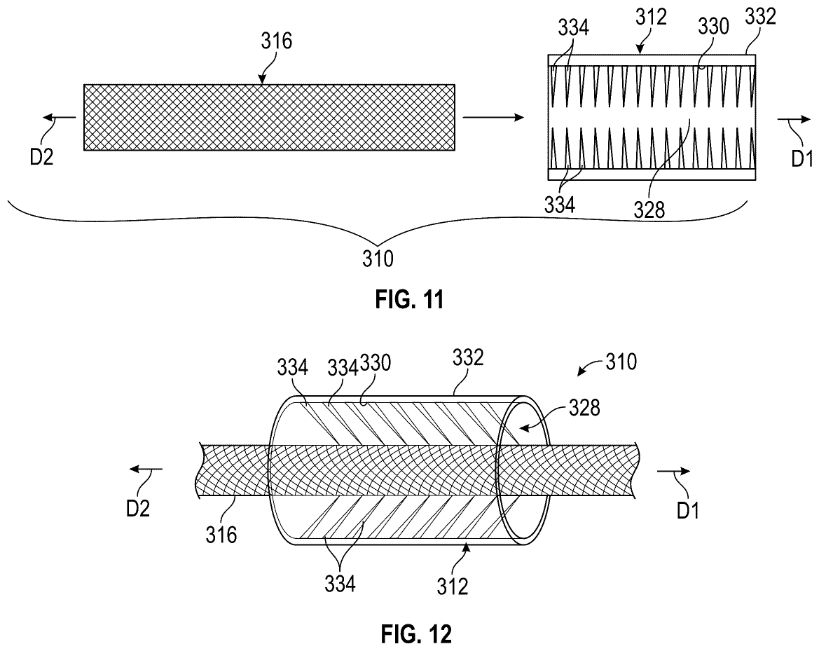

FIGS. 11 and 12 illustrate yet another exemplary suture locking system 310 that can be utilized to perform various tensionable knotless surgical procedures. The suture locking system 310 may include a suture sheath 312 and at least one suture 316. The suture sheath 312 may be a “soft” body made exclusively of soft, suture-based materials and may include an inner diameter wall 330 and an outer diameter wall 332.

A cannulation 328 may extend through the suture sheath 312. The cannulation 328 may establish an internal passageway for accommodating one or more strands of the suture 316. The cannulation 328 may be circumscribed by the inner diameter wall 330.

A plurality of directional fibers 334 may protrude inwardly from the inner diameter wall 330 of the suture sheath 312. The directional fibers 334 may therefore occupy at least a portion of the open space of the cannulation 328. The directional fibers 334 may be made of a suture material or some other compatible material. In an embodiment, the directional fibers 334 are made of a material that is less stiff/rigid than the suture sheath 312. The directional fibers 334 may therefore be configured to flex relative to the suture sheath 312 as the suture 316 is pulled in a first direction D1 through the cannulation.

The directional fibers 334 may therefore establish a one-way locking mechanism that permits the suture 316 to pass through the cannulation 328 in the first direction D1 while preventing the suture 316 from being tensioned or otherwise moved in a second direction D2. For example, the directional fibers 334 may flex to the angled position shown in FIG. 12 as the suture 316 is loaded through the cannulation 328 of the suture sheath 312, and any subsequent tension applied in the second direction D2 may cause the directional fibers 334 to interdigitate with the section of the suture 316 accommodated within the cannulation 328, thereby locking the suture 316 in place and preventing it from sliding back toward the second direction D2.

The suture locking systems of this disclosure may be utilized for performing various tensionable knotless surgical repairs. The suture locking systems provide for tensioning and retensioning suture(s) at various points of the repair, including subsequent to implantation of accompanying fixation devices, thus providing numerous advantages over prior tissue repair systems and techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A suture locking system, comprising:
a first bone tunnel cap;
a second bone tunnel cap configured to engage the first bone tunnel cap;
a one-way locking mechanism provided within a cannulation of the first bone tunnel cap, the second bone tunnel cap, or both; and
a suture received through the first bone tunnel cap and the second bone tunnel cap, wherein the one-way locking mechanism is configured to allow the suture to be tensioned in a first direction while preventing the suture from moving in a second direction.

2. The suture locking system as recited in claim 1, wherein the one-way locking mechanism is established by a plurality of locking barbs provided within a cannulation of at least one of the first bone tunnel cap or the second bone tunnel cap.

3. The suture locking system as recited in claim 1, wherein the first bone tunnel cap and the second bone tunnel cap each include a cap portion and a tube portion that protrudes from the cap portion.

4. The suture locking system as recited in claim 3, wherein the tube portion of the first bone tunnel cap or the second bone tunnel cap is accommodated within the tube portion of the other of the first bone tunnel cap or the second bone tunnel cap.

5. The suture locking system as recited in claim 1, wherein the one-way locking mechanism includes a first one-way locking mechanism provided within the first bone tunnel cap and a second one-way locking mechanism provided within the second bone tunnel cap.

6. The suture locking system as recited in claim 1, comprising a hydroxyapatite coating applied on select portions of the first bone tunnel cap and the second bone tunnel cap.

7. A suture locking system, comprising:
a locking sleeve or a locking screw having an inner diameter wall and an outer diameter wall;
a bone engagement feature provided on the outer diameter wall;
a plurality of locking barbs protruding inwardly from the inner diameter wall and arranged to establish a one-way locking mechanism; and
a suture received through the locking sleeve or the locking screw, wherein the one-way locking mechanism is configured to allow the suture to be tensioned in a first direction and prevent the suture from moving in a second direction.

8. The suture locking system as recited in claim 7, wherein the bone engagement feature includes a thread.

9. The suture locking system as recited in claim 7, wherein the suture is connected to a button.

10. The suture locking system as recited in claim 7, wherein the suture includes an eyelet.

11. The suture locking system as recited in claim 7, wherein the suture includes a suture loop.

12. The suture locking system as recited in claim 7, comprising a hydroxyapatite coating applied on select portions of the outer diameter wall of the locking sleeve or the locking screw.

13. The suture locking system as recited in claim 7, wherein the plurality of locking barbs extend into a cannulation of the locking sleeve or the locking screw.

14. The suture locking system as recited in claim 13, wherein the inner diameter wall circumscribes the cannulation.

15. A suture locking system, comprising:
a suture sheath;
a plurality of directional fibers arranged to establish a one-way locking mechanism within a cannulation of the suture sheath; and
a suture received through the cannulation.

16. The suture locking system as recited in claim 15, wherein the one-way locking mechanism allows the suture to be tensioned in a first direction and prevents the suture from moving in a second direction.

17. The suture locking system as recited in claim 15, wherein the plurality of directional fibers are configured to flex relative to the suture sheath.

18. The suture locking system as recited in claim 15, wherein the suture sheath includes an inner diameter wall that circumscribes the cannulation.

19. The suture locking system as recited in claim 18, wherein the plurality of directional fibers protrude inwardly from the inner diameter wall.

20. The suture locking system as recited in claim 15, wherein the suture sheath includes a first stiffness, and the plurality of directional fibers include a second stiffness that is less than the first stiffness.
